(19) United States
(12) Patent Application Publication
(10) Pub. No.: US 2026/0354892 A1
(43) Pub. Date: Nov. 12, 2026

(54) DETECTION OF ABLATION CATHETER FAILURES

(71) Applicant: Medtronic, Inc., MINNEAPOLIS, MN (US)

(72) Inventors: Matthew J. Hoffman, St. Paul, MN (US); Jeffrey D. Wilkinson, Vadnais Heights, MN (US)

(21) Appl. No.: 19/130,706

(22) PCT Filed: Oct. 31, 2023
(86) PCT No.: PCT/IB2023/060973

§ 371 Date: May 16, 2025

Related U.S. Application Data
(60) Provisional application No. 63/384,903, filed on Nov. 23, 2022.

(51) Int. Cl. A61B 18/14; A61B 18/00

(52) U.S. Cl. CPC A61B 18/1492; A61B 2018/00613; A61B 2018/00642; A61B 2018/00898; A61B 2560/0276

(57) ABSTRACT

An example method for accessing catheter integrity includes outputting, by a device and onto a first conductor of a plurality of conductors of a catheter for use in performing irreversible electroporation (IRE) of target tissue of a patient, a pulse of electrical energy; measuring, by the device and at the first conductor, a reflected electrical energy signal resulting from the pulse of electrical energy; determining, by the device and based on the measured reflected electrical energy signal, a health statistic of the catheter; and outputting, by the device, a representation of the statistic.

This application claims the benefit of U.S. Provisional Patent Application Ser. No. 63/384,903, filed Nov. 23, 2022, the entire content of which is incorporated herein by reference.

### TECHNICAL FIELD

The present technology is related to ablation catheters. In particular, various examples of the present technology are related to detection of failures in ablation catheters.

### BACKGROUND

Tissue ablation is a medical procedure commonly used to treat conditions such as cardiac arrhythmias, which includes atrial fibrillation. For treating cardiac arrhythmias, ablation can be performed to modify tissue, such as to stop aberrant electrical propagation and/or disrupt aberrant electrical conduction through cardiac tissue. Ablation techniques include irreversible electroporation (IRE), cryoablation, laser ablation, thermal ablation, radioablation and radiofrequency (RF) ablation.

Cardiac arrhythmias are a group of conditions that cause an irregular heartbeat or conduction pattern. Ablation may be used to create a safe and effective lesion or set of lesions at the origin of the irregular heartbeat or in regions that aid in the termination of arrhythmias without causing damage to adjacent structures or surrounding tissue, ideally resulting in no need for a maintenance treatment regimen, such as medications or cardioversions.

### SUMMARY

The present technology is directed to devices, systems, and methods for detecting faults in ablation catheters. Irreversible electroporation (IRE), such as Pulsed Field Ablation (PFA), is an ablation therapy that irreversibly electroporates cardiac tissue through the application of pulsed electric fields, with the energy being delivered via PFA ablation catheters. The energy used to create transmural lesions can be in excess of 1500V. As such, imperfections or faults in the conductors or insulation of a PFA ablation catheter or associated cabling, or connectivity failures between system components, could result in undesirable events during delivery of ablation therapy via the PFA ablation catheter (e.g., sub-optimal therapy).

In accordance with one or more aspects of this disclosure, a device may perform one or more operations to automatically detect faults in a PFA ablation catheter. For instance, a device may output a signal onto a conductor of a PFA ablation catheter and measure a reflected signal resulting from the output signal. The device may detect faults in the PFA ablation catheter based on the reflected signal (e.g., using time domain reflectometry). For instance, as changes in impedance cause changes in a fraction of the signal that is reflected, artifacts in the reflected signal may indicate faults (e.g., open circuits) in the conductor. In this way, a device may detect faults in a PFA ablation catheter.

In one example, a method for accessing catheter integrity outputting, by a device and onto a first conductor of a plurality of conductors of a catheter for use in performing PFA of target tissue of a patient, a pulse of electrical energy; measuring, by the device and at the first conductor, a reflected electrical energy signal resulting from the pulse of electrical energy; determining, by the device and based on the measured reflected electrical energy signal, a health statistic of the catheter; and outputting, by the device, a representation of the statistic.

In another example, a device includes a memory; a signal generator; and processing circuitry configured to cause the signal generator to output, onto a first conductor of a plurality of conductors of a catheter for use in performing PFA of target tissue of a patient, a pulse of electrical energy; receive, at the first conductor, a reflected electrical energy signal resulting from the pulse of electrical energy; determine, by the device and based on the received reflected electrical energy signal, a health statistic of the catheter; and output a representation of the statistic.

In another example, a non-transitory computer-readable storage medium stores instructions that, when executed cause processing circuitry to cause a signal generator to output, onto a first conductor of a plurality of conductors of a catheter for use in performing PFA of target tissue of a patient, a pulse of electrical energy; receive, at the first conductor, a reflected electrical energy signal resulting from the pulse of electrical energy; determine, by the device and based on the received reflected electrical energy signal, a health statistic of the catheter; and output a representation of the statistic.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a conceptual diagram illustrating an example system for delivering ablation and detecting catheter failures, in accordance with one or more aspects of this disclosure.

FIG. 2 is a block diagram illustrating an example controller of an ablation system, in accordance with one or more aspects of this disclosure.

FIGS. 3A and 3B are graphs illustrating example measured reflected signals from a catheter, in accordance with one or more aspects of this disclosure.

FIG. 4 is a flowchart illustrating an example ablation catheter health assessment technique, in accordance with one or more techniques of this disclosure.

### DETAILED DESCRIPTION

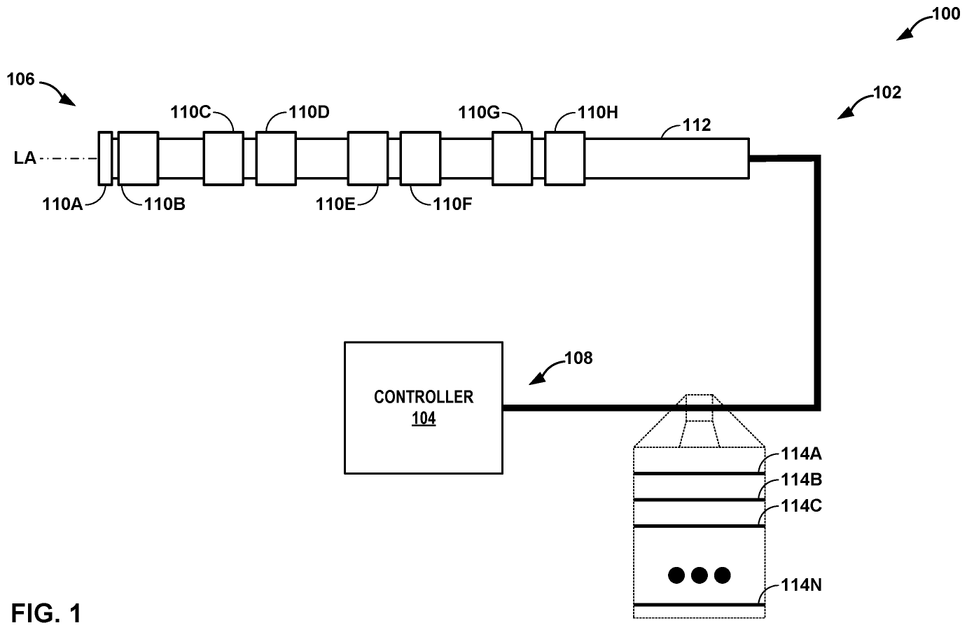

FIG. 1 is a conceptual diagram illustrating an example system for delivering ablation and detecting catheter failures, in accordance with one or more aspects of this disclosure. System 100 includes a catheter 102 and a controller 104. In general, to deliver ablation, a practitioner (e.g., electrophysiologist, interventional cardiologist, etc.) may insert one or more of catheter 102 into a patient and cause controller 104 to deliver, via catheter 102, energy (e.g., pulsed field ablation energy, radiofrequency ablation energy, and the like) to target tissue of a patient. Ablation may cause lesions in target cardiac tissue which may mitigate or stop cardiac arrhythmias. As ablation causes lesions in the heart, it may be desirable for a system to safely and accurately provide information to a clinician regarding ablation parameters, such as a suggested location where to position one or more energy delivery elements of one or more ablation catheters, an amount of energy to be delivered during ablation, or which tissue would be ablated after delivery of ablation energy via the at least one energy delivery element.

Catheter 102 may include elongated structure 112 carrying a plurality of energy delivery elements 110A-110H (collectively “energy delivery elements 110”). An energy delivery element may include an electrode (e.g., in the case of a pulsed field ablation catheter), a radiofrequency element (e.g., in the case of a radiofrequency ablation catheter), or another energy delivery element. While the techniques of this disclosure are applicable to any ablation catheter, the example of FIG. 1 is directed to a pulsed field ablation catheter. Catheter 102 may generally include features that enable insertion of catheter 102 into a patient and navigation of catheter 102 to a target tissue site. Elongated structure 112 may include a distal portion 106 and a proximal portion 108. Energy delivery elements 110 may be generally positioned at distal portion 106, while proximal portion 108 may be connected to controller 104. Energy delivery elements 110 may be of any suitable geometry. Example geometries of electrodes include, but are not necessarily limited to, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes). Energy delivery elements 110 may be axially distributed along longitudinal axis LA of elongated structure 112 or in several other configurations. In some examples, catheter 102 may include one or more energy delivery elements 110 and the geometry of the one or more energy delivery elements 110 may include a balloon, which may be inflated when performing ablation and deflated when navigating catheter 102 to the target tissue. The delivery elements 110 may also be in a circular form, in an array, along multiple splines, or in other configurations.

Elongated structure 112 may include conductors 114A-114N (collectively, “conductors 114”) that may be configured to carry electrical signals between energy delivery elements 110 and controller 104 (shown in an expanded view). Examples of conductors 114 include, but are not necessarily limited to, wires (e.g., solid, braded, etc.), traces, and the like. In some examples, elongated structure 112 may include a separate conductor of conductors 114 for each of energy delivery elements 110. For instance, in the example of FIG. 1 where energy delivery elements 110 includes eight electrodes, elongated structure 112 may include eight separate conductors (e.g., conductor 114A may carry electrical signals for energy delivery element 110A, conductor 114B may carry electrical signals for energy delivery element 110B, . . . , and conductor 114H may carry electrical signals for energy delivery element 110H. In this way, elongated structure may enable each electrode of energy delivery elements 110 to be driven with a different signal from controller 104. In other examples, multiple electrodes of energy delivery elements 110 may share a common conductor. For instance, energy delivery elements 110C and 110D may be connected to a same (e.g., a common) conductor of conductors 114. While such a common conductor arrangement may reduce energy delivery element flexibility (e.g., as electrodes connected to the common conductor may be driven with a same signal), such an arrangement may reduce manufacturing complexity and/or cost and may increase the structural flexibility of catheter 102.

In some examples, catheter 102 may be directly connected to controller 104. In other examples, catheter 102 may be connected to controller 104 via cabling. For instance, a cable may connect to a connector of catheter 102 and to a connector of controller 104. The cabling may include conductors configured to transfer electrical energy between controller 104 and catheter 102.

As shown in FIG. 1, energy delivery elements 110 may include a tip electrode (e.g., electrode 110A), which may be a ring electrode with a “cap” covering at least a portion of a tip of elongated structure 112. In some examples, the tip electrode may be chamfered or otherwise rounded (e.g., to enable easier passage of catheter 102 through anatomy of the patient). Energy delivery elements 110 may include a ring electrode (e.g., electrode 110B) that is adjacent to the tip electrode. This ring electrode may be separated (axially along LA) from the tip electrode. Energy delivery elements 110 may include one or more pairs of ring electrodes. A pair of ring electrodes may include two adjacently closely spaced electrodes of energy delivery elements 110. For instance, in the example of FIG. 1, energy delivery elements 110C and 110D may form a first pair of ring electrodes, energy delivery elements 110E and 110F may form a second pair of ring electrodes, and energy delivery elements 110G and 110H may form a third pair of ring electrodes. In general, the first pair of ring electrodes (i.e., energy delivery elements 110C and 110D) may be accompanied by one or more additional electrodes. The one or more additional electrodes may include any combination of pairs of ring electrodes and coil electrodes (e.g., electrodes that include conductors that spiral around elongated structure 112).

In the example of FIG. 1, energy delivery elements 110 are illustrated as has having a larger diameter than elongated structure 112. In some examples, one or more of energy delivery elements 110 may have a diameter that is approximately equal to or less than the diameter of elongated structure 112. For instance, energy delivery elements 110 may be recessed in elongated structure 112 such that the combination results in a relatively smooth outer surface.

Controller 104 may include an energy generator configured to provide electrical pulses to energy delivery elements 110 to perform an ablation procedure to cardiac tissue or other tissues within the patient's body, such as renal tissue, airway tissue, and organs or tissue within the cardiac space or the pericardial space. For instance, the energy generator may be configured and programmed to deliver pulsed, high-voltage electric fields appropriate for achieving desired pulsed, high-voltage ablation (referred to as “pulsed field ablation” or “pulsed electric field ablation”) and/or pulsed radiofrequency ablation.

As discussed above, conductors 114 may carry electrical signals between controller 104 and energy delivery elements 110. Due to various circumstances, one or more of conductors 114, cabling, or other components may become damaged (e.g., improper handling). Such imperfections or faults could result in undesirable events during delivery of energy (e.g., PFA ablation therapy) via catheter 102. As such, it may be desirable to determine whether catheter 102 (or associated cabling) has any imperfections or faults.

In accordance with one or more aspects of this disclosure, controller 104 may perform one or more operations to automatically detect faults in catheter 102. For instance, controller 104 may output a signal onto a conductor of conductors 114 of catheter 102 and measure a reflected signal resulting from the output signal. Controller 104 may detect faults in catheter 102 based on the reflected signal (e.g., using time domain reflectometry).

A signal incident on a conductor will propagate along it at a large fraction of the speed of light, with the velocity determined by the impedance characteristics of the conductor. When the propagating signal encounters a change in impedance a fraction of the signal continues to propagate in the forward direction and the remaining energy is reflected in the reverse direction. The time course of the signal at the source end of the conductor reveals the magnitude of the impedance changes and, according to their time delay from the start of the source pulse, their location along the conductor. Abrupt changes in impedance, such as caused by an open or shorted conductor, result in large increases and decreases in the detected amplitude, respectively. As such, by monitoring the signal at the source end of the conductor of conductors 114, controller 104 may detect these changes and localize their position along the conductor of conductors 114.

Furthermore, in some examples, controller 104 may detect more subtle variations in impedance, such as poor connections or stressed conductors, via further analysis of the detected waveform. In this way, controller 104 may detect faults in catheter 102.

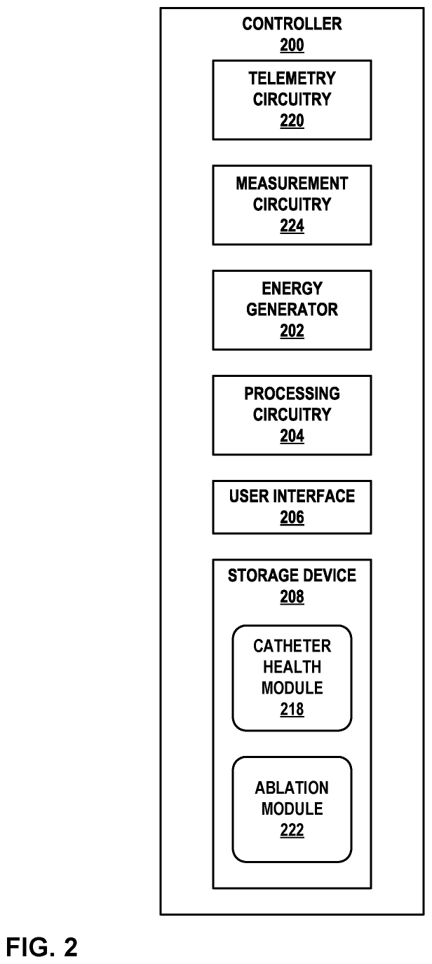

FIG. 2 is a block diagram illustrating an example controller of an ablation system, in accordance with one or more aspects of this disclosure. Controller 200 of FIG. 2 may be an example of controller 104 of FIG. 1. As shown in FIG. 2, controller 200 may include energy generator 202, processing circuitry 204, user interface 206, and storage device 208.

Energy generator 202 may be configured to control energy delivery elements 110 of catheter 102 (FIG. 1) such as to provide electrical pulses to electrodes (e.g., energy delivery elements 110) to perform an electroporation procedure or other ablation procedure to cardiac tissue or other tissues within the patient's body, including but not limited to renal tissue, airway tissue, bones, organs, or tissue within the cardiac space or the pericardial space. For instance, energy generator 202 may be configured and programmed to deliver pulsed, high-voltage electric fields appropriate for achieving desired pulsed, high-voltage ablation (referred to as “pulsed field ablation” or “pulsed electric field ablation”) and/or pulsed radiofrequency ablation. As such, energy generator 202 may be referred to as a PFA signal generator. While shown in the example of FIG. 2 as a single energy generator, energy generator 202 is not so limited. For instance, controller 200 may include multiple energy generators that are each capable of generating ablation signals in parallel. In some examples, controller 200 may include energy generators of different types, such as a pulsed field energy generator and/or a radio frequency energy generator.

Processing circuitry 204 may include one or more processors, such as any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 210 herein may be embodied as firmware, hardware, software or any combination thereof. Processing circuitry 204 may execute instructions to control energy generator 202 to generate signals according to various settings (e.g., ablation module 222 to deliver PFA ablation energy). In some examples, processing circuitry 204 may execute other instructions stored in storage device 208 (e.g., catheter health module 218) to perform catheter testing/fault detection.

Storage device 208 may be configured to store information within controller 200, respectively, during operation. Storage device 208 may include a computer-readable storage medium or computer-readable storage device. In some examples, storage device 208 includes one or more of a short-term memory or a long-term memory. Storage device 208 may include, for example, random-access memories (RAM), dynamic random-access memories (DRAM), static random-access memories (SRAM), ferroelectric random-access memories (FRAM), magnetic discs, optical discs, flash memories, or forms of electrically programmable memories (EPROM) or electrically erasable and programmable memories (EEPROM). In some examples, storage device 208 is used to store data indicative of instructions, e.g., for execution by processing circuitry 204, respectively. Storage device 208 may be configured to store catheter health module 218 and/or ablation module 222.

User interface 206 may include a button or keypad, lights, a speaker for voice commands, a display, such as a liquid crystal (LCD), light-emitting diode (LED), or organic light-emitting diode (OLED). In some examples, the display may be configured to display a representation of a catheter health statistic, as described in further detail herein. For example, processing circuitry 204 may execute catheter health module 218 to perform testing of a catheter (e.g., catheter 102 of FIG. 1) and/or associated cabling, and output the results (e.g., present a graphical user interface) via user interface 206.

The display may be used to guide the clinician in positioning the at least one energy delivery element to a position in contact with or near the suggested target tissue such that during the delivery of energy to the at least one energy delivery element, the target tissue is ablated. In some examples, the display may display a representation of the target tissue that will be ablated after delivery of ablation energy via the at least one energy delivery element. Processing circuitry 204 may then output for display to user interface 206 a representation of the tissue that was ablated. In some examples, the display includes a touch screen. User interface 206 may be configured to display any information related to the performance of ablation. User interface 206 may also receive user input (e.g., selection of target tissue) via user interface 206. The user input may be, for example, in the form of pressing a button(s) on a keypad or selecting an icon(s) from a touch screen. User interface 206 may indicate which conductors are available for ablation energy (have good connectivity to the delivery electrode).

Telemetry circuitry 220 may include any suitable hardware, firmware, software or any combination thereof for communicating with another device. Telemetry circuitry 220 may be configured to communicate using any of a variety of wireless communication schemes, such as Bluetooth® or Bluetooth Low Energy®, WiFi, 4G, or 5G, or a wired communication scheme such as ethernet. Under the control of processing circuitry 204, telemetry circuitry 220 may receive downlink telemetry from, and/or send uplink telemetry to one or more external devices, with the aid of an internal or external antenna, or via wires.

Measurement circuitry 224 may include any suitable hardware, firmware, software or any combination thereof for measuring electrical signals from a catheter connected to controller 200. For instance, measurement circuitry 224 may include one or more analog-to-digital converters (ADC) configured to convert analog voltage measurements (e.g., voltage levels of signals measured at proximal ends of conductors of a catheter) into digital values, which may be processed by processing circuitry 204.

As noted above, processing circuitry 204 may execute catheter health module 218 to perform catheter testing/fault detection. To perform the catheter testing/fault detection, processing circuitry 204 may execute catheter health module 218 to cause energy generator 202 to output, onto a proximal end of a first conductor of a plurality of conductors of a catheter for use in performing pulse field ablation (PFA) of target tissue of a patient, a pulse of electrical energy. For instance, energy generator 202 may output the electrical pulse onto a conductor of conductors 114 of catheter 102 of FIG. 1. A return path may be established using one or more other conductors of conductors 114. The pulse of electrical energy may have a voltage level less than N volts (e.g., 50 millivolts, 0.5 volts, 1 volt, 2 volts, 3 volts, 4 volts, 5 volts). In some examples, the pulse may be a square wave pulse. In other examples, more complex signals may be used (e.g., single-cycle or half-cycle sinusoids).

The pulse may propagate along the conductor until reaching any changes in impedance. As discussed above, changes in impedance may result in a fraction of the signal being reflected back to the proximal end of the conductor. Measurement circuitry 224 may measure, at the first conductor, a reflected electrical energy signal resulting from the pulse of electrical energy. For instance, an ADC of measurement circuitry 224 may convert voltage levels at the proximal end of the first conductor into a stream of digital values, and provide the digital values to processing circuitry 204.

Controller 200 may determine, based on the measured reflected electrical energy signal, a health statistic of the catheter. In general, a health statistic is a measurement representative of a viability of conductors. For instance, processing circuitry 204 may execute catheter health module 218 to analyze the digital values to determine the health statistic, e.g., whether the conductor has any faults. In some cases (e.g., under low noise conditions) controller 200 may utilize a single pulse to perform the catheter testing/fault detection. In other cases, controller 200 may utilize a plurality of pulses (and their corresponding return signals) to perform the catheter testing/fault detection (e.g., the pulse may be periodically repeated and measured so that signal averaging or other processing steps may be utilized to improve detection accuracy).

To determine the health statistic based on the reflected electrical energy signal, controller 200 may determine a temporal displacement between a first time at which the pulse of electrical energy is output and a second time at which the reflected electrical energy signal is received. The temporal displacement between the first time and the second time may be indicative of a location of a change in impedance along the catheter. As changes in impedance may be the result of faults or other damage, the temporal displacement thereby represents a location of the fault along the catheter (e.g., determined based on a propagation speed of the signal). In some examples, controller 200 may use template matching of morphologies (e.g., templates of “good” and “bad” states) to determine presence of faults.

Controller 200 may determine the health statistic to indicate that the catheter has faults or does not have faults. Where controller 200 determines the health statistic as indicating that the catheter has one or more faults, the health statistic may indicate a location of the fault on the catheter (e.g., 30 cm from the proximal end).

In some examples, controller 200 may perform the signal output and measurement on a single conductor of the catheter. In other examples, controller 200 may perform the signal output and measurement on a plurality of conductors of the catheter. For instance, controller 200 may output a respective pulse of the plurality of pulses onto respective conductor of the plurality of conductors, and measure, at each respective conductor of the plurality of conductors, a respective reflected energy signal of a plurality of reflected energy signals. As such, in some examples, controller 200 may determine a single health statistic that represents all the conductors (e.g., determine the health statistic of the catheter based on the plurality of reflected energy signals).

Controller 200 may output a representation of the statistic. As one example, processing circuitry 204 may cause user interface 206 to display a binary representation of whether the catheter has a fault or not. As another example, processing circuitry 204 may cause user interface 206 to display a graphical user interface (GUI) that indicates whether the catheter has a fault or not, which conductors of the catheter have faults, and/or a location of the faults.

In some examples, in addition to or in place of outputting the representation of the statistic, controller 200 may perform one or more actions. As one example, responsive to the health statistic of the catheter indicating a fault in the catheter, controller 200 may prevent the delivery of ablation energy (e.g., PFA ablation energy) via the catheter. For instance, catheter health module 218 may prevent ablation module 222 from causing energy generator 202 from outputting the ablation energy via the catheter.

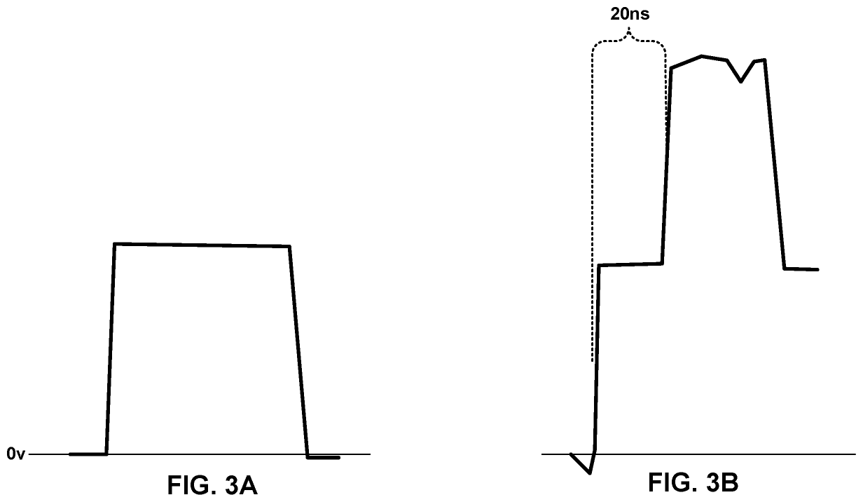

FIGS. 3A and 3B are graphs illustrating example measured reflected signals from a catheter, in accordance with one or more aspects of this disclosure. FIG. 3A illustrates an example reflected signal from a conductor of a catheter with an impedance matched termination at a distal end. For instance, FIG. 3A illustrates a measured reflected signal from a 2 meter (m), 50-ohm coax cable with a 50-ohm termination at its distal end that is stimulated with a 10 MHz square wave with a 30 picosecond (ps) rise time. The flat top of the signal of FIG. 3A. indicates no impedance variation along the length of the conductor. FIG. 3B illustrates an example reflected signal from a conductor of a catheter with an open distal end. For instance, FIG. 3B illustrates a measured reflected signal from the same cable as FIG. 3A, but with the distal end unterminated. As can be seen in FIG. 3B, there is an abrupt jump (doubling) in amplitude after 20 nanoseconds (ns), corresponding to the roundtrip propagation time for a 2 m cable. If the conductor being stimulated were open halfway along its length the doubling would occur at 10 nanoseconds, signaling the fault condition at that location (i.e., at the halfway location).

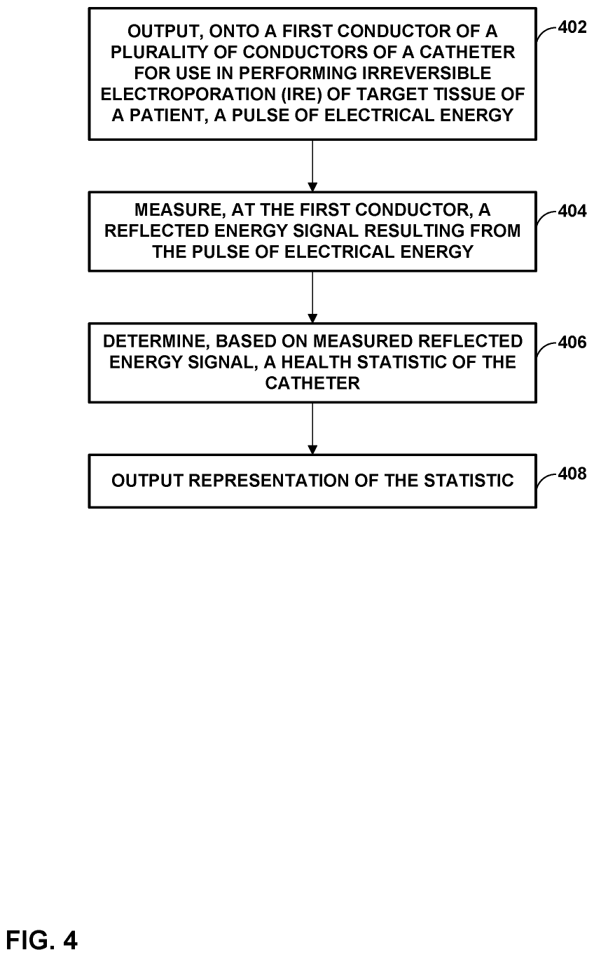

FIG. 4 is a flowchart illustrating an example ablation catheter health assessment technique, in accordance with one or more techniques of this disclosure. The technique of FIG. 4 may be performed by a controller, such as controller 104 of FIG. 1 or controller 200 of FIG. 2.

Controller 200 may output, onto a first conductor of a plurality of conductors of a catheter for use in performing irreversible electroporation (IRE), such as pulse field ablation (PFA), of target tissue of a patient, a pulse of electrical energy (402). For instance, controller 104 may cause a signal generator to output a pulse of electrical energy onto a proximal end of conductor 114A (e.g., while a distal end of conductor 114A, or an electrode connected thereto, is connected to one or more other conductors of conductors 114, or electrodes connected thereto).

Controller 200 may measure, at the first conductor, a reflected electrical energy signal resulting from the pulse of electrical energy (404). For instance, controller 104 may sense voltage levels at the proximal end of conductor 114A. As noted above, an ADC may convert the sensed voltage levels into digital values.

Controller 200 may determine, based on the measured reflected electrical energy signal, a health statistic of the catheter (406). For instance, controller 104 may perform time domain reflectometry (TDR) to analyze the measured reflected electrical energy signal and determine whether there are any faults along conductor 114A. The health statistic may indicate whether any faults were detected by controller 104. As discussed above, controller 104 may perform TDR on multiple conductors of the catheter, or may perform TDR on a single conductor of the catheter. As such, the statistic may represent a health of one or more of the conductors.

Controller 200 may output a representation of the statistic (408). As one example, controller 104 may cause a user 206 to display a binary representation of whether the catheter has a fault or not. As another example, controller 104 may cause the user interface to display a graphical user interface (GUI) that indicates whether the catheter has a fault or not, which conductors of the catheter have faults, and/or a location of the faults.

Controller 200 may perform the ablation catheter health assessment at one or more points in the catheter lifecycle. As one example, controller 200 may perform the ablation catheter health assessment after the system components are connected, which may be referred to as a system connection check (e.g., to assess connectivity). As another example, controller 200 may perform the ablation catheter health assessment after the catheter is placed, which may be referred to as a conductor break check (e.g., to assess whether or not an effective therapy is likely to occur or if there is an issue with a catheter interface cable (CIC) (e.g., a cable that connects a proximal end of the catheter to the controller) that can be remedied without having to remove the catheter). As another example, controller 200 may perform the ablation catheter health assessment after the therapy delivery is attempted, which may be referred to as a therapy integrity check (e.g., in order to determine if the conductors were damaged during the therapy delivery).

For the system connection check, controller 104 may individually connect the pulse source to each electrode terminal (e.g., each conductor) in turn. All other terminals may be connected together and are used as a signal return path. As noted above, an abrupt rise in the signal amplitude indicates an open condition and its occurrence time relative to the input signal indicates its location. Analysis of the signal would confirm connections from the generator to the distal end of the cable. A disconnected CIC may result in an early doubling of the signal amplitude. Similarly, a disconnected catheter may be revealed by an increase in the signal amplitude (e.g., after an additional 25 ns). This check may be made prior to the catheter being inserted into the patient or while the catheter is in place. Responsive to detecting a failure, controller 104 may output a system warning that instructs a user on how to mitigate issues, or prevent delivery of therapy via the compromised conductor/electrode. This may also be a mechanism to allow system compatibility of the signal generator with catheter designs with ‘less than the maximum allowed’ electrodes (restricting energy delivery to only connected conductors/electrodes). As such, a procedure that would otherwise have to be canceled may continue with reduced functionality.

For the conductor break check, controller 104 may, using the same measurement scheme as above, may determine a location of a broken (open) conductor based on a rise in the signal amplitude. By examining the time between the source edge and the rise, controller 104 may not only diagnose the presence of a broken connection but to localize it to the CIC or catheter. A short between a conductor and one or more of its neighboring conductors may be detectable based on an abrupt decrease in the signal amplitude. Controller 104 may perform this check may be conducted with the prior to catheter insertion or while it is in place. Performing the check may be particularly valuable once the catheter is in place as it could detect a break in the CIC (e.g., which could be remedied without removing the catheter). This procedure may also assure patent connections, enabling effective delivery of therapy.

For the therapy integrity check, controller 104 may, using the same measurement scheme as above, perform an assessment of conductivity after the therapy energy is delivered. As such, controller 104 may determine if the conductors were damaged during the therapy delivery. If the conductors were damaged during the therapy delivery, effective therapy may not be guaranteed.

The techniques described in this disclosure may be implemented, at least in part, in hardware, software, firmware or any combination thereof. For example, various aspects of the described techniques may be implemented within processing circuitry, which may include one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also form one or more processors or processing circuitry configured to perform one or more of the techniques of this disclosure.

Such hardware, software, and firmware may be implemented, and various operation may be performed within same device, within separate devices, and/or on a coordinated basis within, among or across several devices, to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components. Processing circuitry described in this disclosure, including a processor or multiple processors, may be implemented, in various examples, as fixed-function circuits, programmable circuits, or a combination thereof. Fixed-function circuits refer to circuits that provide particular functionality with preset operations. Programmable circuits refer to circuits that can be programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive stimulation parameters or output stimulation parameters), but the types of operations that the fixed-function circuits perform are generally immutable. In some examples, one or more of the units may be distinct circuit blocks (fixed-function or programmable), and in some examples, one or more of the units may be integrated circuits.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

The following examples are a non-limiting list of clauses in accordance with one or more techniques of this disclosure.

Example 1. A method for accessing catheter integrity, the method comprising:
outputting, by a device and onto a first conductor of a plurality of conductors of a catheter for use in performing irreversible electroporation (IRE) of target tissue of a patient, a pulse of electrical energy; measuring, by the device and at the first conductor, a reflected electrical energy signal resulting from the pulse of electrical energy; determining, by the device and based on the measured reflected electrical energy signal, a health statistic of the catheter; and outputting, by the device, a representation of the statistic.

Example 2. The method of Example 1, wherein the device is an IRE signal generator.

Example 3. The method of Example 1, wherein the device does not include an IRE signal generator.

Example 4. The method of any of Examples 1-3, wherein outputting the pulse onto the first conductor comprises outputting a first pulse of a plurality of pulses onto the first conductor, the method further comprising: outputting, by the device, a respective pulse of the plurality of pulses onto respective conductor of the plurality of conductors; and measuring, by the device and at each respective conductor of the plurality of conductors, a respective reflected energy signal of a plurality of reflected energy signals, wherein determining the health statistic of the catheter comprises determining the health statistic of the catheter based on the plurality of reflected energy signals.

Example 5. The method of Example 4, wherein the health statistic of the catheter includes a respective health statistic of each of the plurality of conductors such that determining the health statistic comprises determining a plurality of health statistics.

Example 6. The method of any of Examples 1-5, wherein accessing the catheter integrity comprises accessing the catheter integrity prior to delivery of IRE ablation energy to a patient via the catheter.

Example 7. The method of Example 6, wherein accessing the catheter integrity prior to delivery of IRE ablation energy to the patient via the catheter comprises accessing the catheter integrity after insertion of the catheter into the patient but prior to the delivery of IRE ablation energy.

Example 8. The method of Example 6 or Example 7, further comprising: responsive to the health statistic of the catheter indicating a fault in the catheter, preventing the delivery of the IRE ablation energy via the catheter.

Example 9. The method of any of Examples 1-5, wherein accessing the catheter integrity comprises accessing the catheter integrity after delivering IRE ablation energy to a patient via the catheter.

Example 10. The method of any of Examples 1-9, wherein outputting the pulse of electrical energy comprises outputting the pulse of electrical energy with a voltage level less than 5 volts.

Example 11. The method of any of Examples 1-10, further comprising: determining a location of a fault between the pulse generating device and delivery electrodes of the catheter based on a temporal displacement between a first time at which the pulse of electrical energy is output and a second time at which the reflected electrical energy signal is received, wherein the health statistic indicates the determined location of the fault.

Example 12. The method of any of Examples 1-11, wherein the catheter is connected to the device via cabling, and wherein the health statistic includes a health statistic of the cabling.

Example 13. A device comprising: a memory; a signal generator; and processing circuitry configured to perform the method of any of Examples 1-12.

Example 14. A computer-readable storage medium storing instructions that, when executed, cause a device to perform the method of any of Examples 1-12.

1. A method for assessing catheter integrity, the method comprising:
outputting, by a device and onto a first conductor of a plurality of conductors of a catheter for use in performing irreversible electroporation (IRE) of target tissue of a patient, a pulse of electrical energy;
measuring, by the device and at the first conductor, a reflected electrical energy signal resulting from the pulse of electrical energy;
determining, by the device and based on the reflected electrical energy signal, a health statistic of the catheter; and
outputting, by the device, a representation of the statistic.

2. The method of claim 1, wherein the device is an IRE signal generator.

3. The method of claim 1, wherein the device does not include an IRE signal generator.

4. The method of claim 1, wherein outputting the pulse onto the first conductor comprises outputting a first pulse of a plurality of pulses onto the first conductor, the method further comprising:
outputting, by the device, a respective pulse of the plurality of pulses onto respective conductor of the plurality of conductors; and
measuring, by the device and at each respective conductor of the plurality of conductors, a respective reflected energy signal of a plurality of reflected energy signals,
wherein determining the health statistic of the catheter comprises determining the health statistic of the catheter based on the plurality of reflected energy signals.

5. The method of claim 4, wherein the health statistic of the catheter includes a respective health statistic of each of the plurality of conductors such that determining the health statistic comprises determining a plurality of health statistics.

6. The method of claim 1, wherein assessing the catheter integrity comprises assessing the catheter integrity prior to delivery of IRE ablation energy to a patient via the catheter.

7. The method of claim 6, wherein assessing the catheter integrity prior to delivery of IRE ablation energy to the patient via the catheter comprises assessing the catheter integrity after insertion of the catheter into the patient but prior to the delivery of IRE ablation energy.

8. The method of claim 6, further comprising:
responsive to the health statistic of the catheter indicating a fault in the catheter, preventing the delivery of the IRE ablation energy via the catheter.

9. The method of claim 1, wherein assessing the catheter integrity comprises accessing the catheter integrity after delivering IRE ablation energy to a patient via the catheter.

10. The method of claim 1, wherein outputting the pulse of electrical energy comprises outputting the pulse of electrical energy with a voltage level less than 5 volts.

11. The method of claim 1, further comprising:
determining a location of a fault between the pulse generating device and delivery electrodes of the catheter based on a temporal displacement between a first time at which the pulse of electrical energy is output and a second time at which the reflected electrical energy signal is received,
wherein the health statistic indicates the location of the fault.

12. The method of claim 1, wherein the catheter is connected to the device via cabling, and wherein the health statistic includes a health statistic of the cabling.

13. A device for assessing catheter integrity, the device comprising:
a memory;
a signal generator; and
processing circuitry configured to assess integrity of a catheter for use in performing irreversible electroporation (IRE) of target tissue of a patient, wherein to assess the integrity of a catheter, the processing circuitry is configured to:
cause the signal generator to output, onto a first conductor of a plurality of conductors of the catheter, a pulse of electrical energy;
measure, at the first conductor, a reflected electrical energy signal resulting from the pulse of electrical energy;
determine, based on the reflected electrical energy signal, a health statistic of the catheter; and
output a representation of the statistic.

14. A computer-readable storage medium storing instructions that, when executed, cause a device to:
cause a signal generator of the device to output, onto a first conductor of a plurality of conductors of a catheter for use in performing irreversible electroporation (IRE) of target tissue of a patient, a pulse of electrical energy;
measure, at the first conductor, a reflected electrical energy signal resulting from the pulse of electrical energy;
determine, based on the reflected electrical energy signal, a health statistic of the catheter; and
output a representation of the statistic.

15. The device of claim 13, wherein, to cause the signal generator to output the pulse onto the first conductor, the processing circuitry is configured to cause the signal generator to output a first pulse of a plurality of pulses onto the first conductor, the processing circuitry further configured to:
cause the signal generator to output a respective pulse of the plurality of pulses onto respective conductor of the plurality of conductors; and
measure, at each respective conductor of the plurality of conductors, a respective reflected energy signal of a plurality of reflected energy signals,
wherein, to determine the health statistic of the catheter, the processing circuity is configured to determine the health statistic of the catheter based on the plurality of reflected energy signals.

16. The device of claim 15, wherein the health statistic of the catheter includes a respective health statistic of each of the plurality of conductors such that determining the health statistic comprises determining a plurality of health statistics.

17. The device of claim 13, wherein, to assess the integrity of the catheter, the processing circuitry is configured to assess the integrity prior to delivery of IRE ablation energy to a patient via the catheter.

18. The device of claim 17, wherein, to assess the catheter integrity prior to delivery of IRE ablation energy to the patient via the catheter, the processing circuitry is configured to assessing the catheter integrity after insertion of the catheter into the patient but prior to the delivery of IRE ablation energy.

19. The device of claim 17, wherein the processing circuitry is further configured to:
prevent, responsive to the health statistic of the catheter indicating a fault in the catheter, the delivery of the IRE ablation energy via the catheter.

20. The device of claim 13, wherein the processing circuitry is further configured to:
determine a location of a fault between the signal generator and delivery electrodes of the catheter based on a temporal displacement between a first time at which the pulse of electrical energy is output and a second time at which the reflected electrical energy signal is received,
wherein the health statistic indicates the location of the fault.
